(19) United States
(12) Patent Application Publication
(10) Pub. No.: US 2017/0315133 A1
(43) Pub. Date: Nov. 2, 2017

(54) METHOD OF DIAGNOSING AND TREATING BRAIN ENDOTHELIAL INFLAMMATION RELATED DISEASE

(71) Applicants: J. Steven ALEXANDER, Shreveport, LA (US); Ikuo TSUNODA, Osaka (JP); Urska CVEK, Shreveport, LA (US); J. Winny YUN, Shreveport, LA (US); Alireza MINAGAR, Shreveport, LA (US)

(72) Inventors: J. Steven ALEXANDER, Shreveport, LA (US); Ikuo TSUNODA, Osaka (JP); Urska CVEK, Shreveport, LA (US); J. Winny YUN, Shreveport, LA (US); Alireza MINAGAR, Shreveport, LA (US)

(73) Assignee: Board of Supervisors of Louisiana State University and Agricultural and Mechanical College, Baton Rouge, LA (US)

(21) Appl. No.: 15/581,626

(22) Filed: Apr. 28, 2017

Related U.S. Application Data
(60) Provisional application No. 62/328,715, filed on Apr. 28, 2016.

(51) Int. Cl. G01N 33/68; G01N 33/564; G01N 33/53; A61K 39/00

(52) U.S. Cl. CPC G01N 33/6863; G01N 33/68; G01N 33/53; G01N 33/564; A61K 39/00; G01N 2800/04; G01N 2800/7085

(57) ABSTRACT

The presently claimed invention relates to materials and methods for diagnosing a chronic inflammatory disease in a patient comprising testing a biological sample from the patient for a level of a first neurovascular biomarker of lymphatic activation and diagnosing the patient as having the chronic inflammatory disease if the tested level of the first neurovascular biomarker of lymphatic activation is less than a first normal level.

### CROSS REFERENCE TO RELATED APPLICATIONS/PRIORITY

The present invention claims priority to U.S. Provisional Patent Application No. 62/328,715 filed Apr. 28, 2016, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND OF THE INVENTION

Multiple Sclerosis (MS) is the leading cause of permanent neurologic disability in young adults. The inventors observe that MS is not just a group of immune-mediated central nervous system (CNS) demyelinating diseases, but one of a group of vascular neuroinflammatory conditions. The inventors offer that the complex pathogenesis of MS can only be appreciated when and if its vascular contributions are recognized as significant features of all forms of MS etiology. The inventors propose that disturbances in the expression and organization of ‘neurolymphatic’/‘glymphatic’ features of blood brain barrier (BBB) vessels represent extremely important and mechanism-based but under-recognized characteristics of the MS inflamed cerebrum that contributed to progression and intensification of the MS with the brain endothelial cells as the ‘failing gatekeeper’. The assumption of the industry has been that the presence of neurolymphatic markers in blood reflected a brain-endothelial origin because of the demonstration of these proteins in human and brain endothelial cells and their supernatants, changes in the abundance and shedding of these proteins following challenge with inflammatory cytokines, the intimate endothelial surface interaction with the circulating serum/plasma and the blood and the initial compartmentalization of these lymphatic biomarkers into ‘microparticles’ (MP).

Because of the long-held perception that the CNS lacks conventional lymphatics, studies on lymphatic involvement in MS pathogenesis have largely neglected roles brain lymphatics might play in eliminating inflammatory mediators in the CNS. The inventors are aware that CNS interstitial fluid and solutes can drain along 150-200 nm wide ‘lacunae’ in the basement membranes of arteries and capillary walls, representing a conduit for elimination of brain interstitial/neurolymphatic contents, and that a brain-wide system of channels facilitate the clearance of interstitial components from the brain which are termed the ‘glymphatic’ system. So far, the molecular identities and cellular origins of these networks remain uncharacterized. The inventors argue that the networks appear to represent important diagnostic, staging and therapeutic targets in neurodegenerative diseases.

Presently, putative MS-patients are only diagnosed with MS after at least two separate areas of the CNS are found to be damaged by MRI imaging. Only then will physicians employ various neurologic exams and lab tests to rule out other diseases to diagnose MS. These lab tests are often complicated and invasive, involving lumbar punctures to obtain and evaluate CSF samples, and forestalls accurate diagnosis until the disease has progressed, increasing severity of the disease. For the foregoing reasons, there is a pressing, but seemingly irresolvable need for accurate, inexpensive, non-obtrusive diagnostic tests for MS, Alzheimer's disease (AD), and other chronic inflammatory diseases.

### SUMMARY OF THE INVENTION

Wherefore, it is an object of the present invention to overcome the above mentioned shortcomings and drawbacks associated with the current technology. The present invention is directed to methods and apparatuses for testing and treating chronic inflammatory diseases, including neuroinflammatory conditions such as MS and AD.

Another object of the present invention is to use MP analysis to evaluate multiple markers of brain inflammation and correlate and validate these findings with MRI or CT imaging.

A further object of the invention is to identify the groupings of MP analyses which yield the most effective PCA interpretation.

A still further object of the invention is expansion of the approach into all circulating microparticle species to overcome drawbacks with single analytes, a major shortcoming of current methodologies.

The present invention relates to methods for testing clinical samples for multiple lymphatic endothelial markers present on MPs. These are derived from the endothelial surface and provide for the first time a liquid ‘snapshot’ of the state of inflammatory activation of the vascular space. This is evidenced to have high predictive value in discriminating disease states and severity

The presently claimed invention relates to materials and methods for diagnosing a chronic inflammatory disease in a patient comprising testing a biological sample from the patient for a level of a first neurovascular biomarker of lymphatic activation and diagnosing the patient as having the chronic inflammatory disease if the tested level of the first neurovascular biomarker of lymphatic activation is less than a first normal level. According to a further embodiment, the chronic inflammatory disease is one of multiple sclerosis, Alzheimer's disease, osteoarthritis, rheumatoid arthritis, lupus, Crohn's disease, ulcerative colitis, graft vs. host disease, transplant injury, and asthma. According to a further embodiment, the chronic inflammatory disease is a brain endothelial related disease. According to a further embodiment, the brain endothelial related disease is one of multiple sclerosis and Alzheimer's disease. According to a further embodiment, the first neurovascular biomarker of lymphatic activation is one of chicken ovalbumin upstream promoter transcription factor II (COUPTF-II), Prospero homeobox transcription factor-1 (Prox-1), Forked winghead transcription factor-2 (Fox2), lymphatic vascular endothelial hyaluronic acid receptor (LYVE-1), vascular endothelial growth factor receptor-3 (VEGFR-3), and podoplanin. A further embodiment relates to testing the biological sample from the patient for a level of a second neurovascular biomarker of lymphatic activation, wherein the second neurovascular biomarker of lymphatic activation is different from the first neurovascular biomarker of lymphatic activation, and diagnosing the patient as having the chronic inflammatory disease if the tested level of both the first neurovascular biomarker of lymphatic activation is less than the first normal level and the second neurovascular biomarker of lymphatic activation is less than a second normal level. According to a further embodiment, the second neurovascular biomarker of lymphatic activation is one of chicken ovalbumin upstream promoter transcription factor II (COUPTF-II), Prospero homeobox transcription factor-1 (Prox-1), Forked winghead transcription factor-2 (Fox2), lymphatic vascular endothelial hyaluronic acid receptor (LYVE-1), vascular endothelial growth factor receptor-3 (VEGFR-3), and podoplanin. A further embodiment comprises the step of diagnosing the patient as having the chronic inflammatory disease if the tested level of either the first neurovascular biomarker of lymphatic activation is less than the first normal level or the second neurovascular biomarker of lymphatic activation is less than the second normal level. A further embodiment comprises testing the biological sample from the patient for a level of a second neurovascular biomarker of lymphatic activation, wherein the second neurovascular biomarker of lymphatic activation is different from the first neurovascular biomarker of lymphatic activation, testing the biological sample from the patient for a level of a third neurovascular biomarker of lymphatic activation, wherein the third neurovascular biomarker of lymphatic activation is different from the first and the second neurovascular biomarker of lymphatic activation, and diagnosing the patient as having the chronic inflammatory disease if the tested level of each of the first neurovascular biomarker of lymphatic activation is less than the first normal level, the second neurovascular biomarker of lymphatic activation is less than a second normal level, and the third neurovascular biomarker of lymphatic activation is less than a third normal level. A further embodiment comprises diagnosing the patient as having the chronic inflammatory disease if the tested level of at least two of the first neurovascular biomarker of lymphatic activation is less than the first normal level, the second neurovascular biomarker of lymphatic activation is less than the second normal level, and the third neurovascular biomarker of lymphatic activation is less than the third normal level. A further embodiment comprises testing the biological sample from the patient for a level of a second neurovascular biomarker of lymphatic activation, wherein the second neurovascular biomarker of lymphatic activation is different from the first neurovascular biomarker of lymphatic activation, testing the biological sample from the patient for a level of a third neurovascular biomarker of lymphatic activation, wherein the third neurovascular biomarker of lymphatic activation is different from the first and the second neurovascular biomarker of lymphatic activation, testing the biological sample from the patient for a level of a fourth neurovascular biomarker of lymphatic activation, wherein the fourth neurovascular biomarker of lymphatic activation is different from the first, the second, and the third neurovascular biomarker of lymphatic activation, and diagnosing the patient as having the chronic inflammatory disease if the tested level of each of the first neurovascular biomarker of lymphatic activation is less than the first normal level, the second neurovascular biomarker of lymphatic activation is less than a second normal level, the third neurovascular biomarker of lymphatic activation is less than a third normal level, and the fourth neurovascular biomarker of lymphatic activation is less than a fourth normal level. A further embodiment comprises diagnosing the patient as having the chronic inflammatory disease if the tested level of at least three of the first neurovascular biomarker of lymphatic activation is less than the first normal level, the second neurovascular biomarker of lymphatic activation is less than the second normal level, the third neurovascular biomarker of lymphatic activation is less than the third normal level, and the fourth neurovascular biomarker of lymphatic activation is less than the fourth normal level. According to a further embodiment, the biological sample is one of blood, serum, plasma, cerebrospinal fluid, saliva, urine, tears. According to a further embodiment, the biological sample is serum.

The presently claimed invention further relates to materials and methods for diagnosing and treating one of a chronic inflammatory disease comprising testing a biological sample from the patient for a level of a first neurovascular biomarker of lymphatic activation, diagnosing the patient as having the chronic inflammatory disease if the tested level of the first neurovascular biomarker of lymphatic activation is less than a first normal level, and administering a therapeutic to the patient for the chronic inflammatory disease. According to a further embodiment, the chronic inflammatory disease is one of multiple sclerosis, Alzheimer's disease, osteoarthritis, rheumatoid arthritis, lupus, Crohn's disease, ulcerative colitis, graft vs. host disease, transplant injury, and asthma. According to a further embodiment, the therapeutic is one of a biologic and an immunotherapy. The therapeutic may be one that is currently known to the art for treating the chronic inflammatory disease. According to a further embodiment, the therapeutic is one of natalizumab, vedolizumab, and steroids.

The presently claimed invention further relates to materials and methods for a kit for carrying out the testing of the biological sample. A further embodiment of the kit includes an immobilizing phase and an anti-first neurovascular biomarker of lymphatic activation antibody.

The inventors have shown, inter alia, that expression changes for the of the lymphatic-lineage specifying transcription factor COUPTF-II (described in FIG. 3 below) as well as the forkhead transcription factor C2 (Fox-C2), and Prospero homeobox-1 (Prox-1)) in both mouse and human brain endothelial cells in response to inflammatory stimuli (tumor necrosis factor-alpha/interferon-g) and the suppression of these markers in MS (described in FIG. 1 below). These proteins affect the expression of neurolymphatic structural proteins (e.g. podoplanin, lymphatic vascular endothelial hyaluronic acid receptor (LYVE-1)), signaling modules (vascular endothelial growth factor-3 (VEGFR-3) and vascular endothelial growth factor-D (VEGF-D). The presence of and changes in these markers in both brain endothelial cells and in plasma/serum from clinical and experimental MS is consistent with these proteins involvement in inflammation, neurodegeneration as well as other forms of chronic inflammation.

The proposed use of multi-marker analysis involving such transcription factors and evaluation of such markers with principal component analysis provides for an unbiased multi-analyte approach based on the use of factor loading for all of the analytes listed above. This approach is powerful, simple and inexpensive.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that while the accompanying graph drawings are to scale, the remainder are not necessarily so, since the emphasis is instead placed on illustrating the principles of the invention. The invention will now be described, by way of example, with reference to the accompanying drawings in which:

FIGS. 1A-1G are a series of blots (FIG. 1A) and six graphs showing the suppression expression of lymphatic biomarkers by brain endothelial cells in response to inflammatory cytokines, with the graphs showing relative expression for the particular biomarker versus the Control, TNFa 20 ng/ml, IFNg 1000 U/ml, and TNF+INF, with the biomarkers including VEGFR-3 (FIG. 1B), Prox-1 (FIG. 1C), FOXC-2 (FIG. 1D), LYVE-1 (FIG. 1E), Podoplanin (FIG. 1F), and Cav-1 (FIG. 1G);

FIG. 2 is a diagram showing the potent suppression of the lymphatic transcription factor COUPTF-II by combined tumor necrosis factor and interferon-gamma exposure;

FIGS. 3A-3H are figures showing the time and cytokine dependent increase in brain endothelial microparticle shedding from the cell surface; FIG. 3A is a graph of MP released following 18 hour treatment, measuring Annexin V+MP for each of control, 1 ng/ml TNF, 10 ng/ml TNF, 20 ng/ml TNF, 10 U/ml IFN, 200 U/ml INF, and 1000 U/ml INF; FIG. 3B is a graph of 18 hour TNF treatment only, measuring MP per ml for each of control, 1 ng/ml TNF, 10 ng/ml TNF and 20 ng/ml TNF; FIG. 3C is a graph of 18 hour IFN treatment only, measuring Annexin V+MP for each of control, 10 U/ml IFN, 200 U/ml INF, and 1000 U/ml INF; FIG. 3D is a graph of D3 MP released following 24 hour treatment, measuring Annexin V+MP for each of control, 1 ng/ml TNF 10 U/ml IFN, 200 U/ml INF, and 1000 U/ml INF; FIG. 3E is a graph of MP released following 42 hour treatment, measuring Annexin V+MP for each of control, 1 ng/ml TNF, 10 ng/ml TNF, 20 ng/ml TNF, 10 U/ml IFN, 200 U/ml INF, and 1000 U/ml INF; FIG. 3F is a graph of 42 hour TNF treatment only, measuring MP per ml for each of control, 1 ng/ml TNF, 10 ng/ml TNF and 20 ng/ml TNF; FIG. 3G is a graph of 42 hour IFN treatment only, measuring Annexin V+MP for each of control, 10 U/ml IFN, 200 U/ml INF, and 1000 U/ml INF; and FIG. 3H is a graph of D3 MP released following 48 hour treatment, measuring Annexin V+MP for each of control, 1 ng/ml TNF 10 U/ml IFN, 200 U/ml INF, and 1000 U/ml INF;

FIG. 4 is a figure with multiple blots showing how microparticles liberate these NEBULA markers into a soluble pool in serum;

FIG. 5 is a diagram which schematically explains FIG. 4;

FIG. 6 is a box plot which shows how COUPTF-II analysis reveals suppression of COUPTF-II expression between healthy controls and MS patients in aggregate;

FIG. 7 is a box plot which shows how COUPTF-II analysis reveals suppression of COUPTF-II expression between healthy controls and relapsing and remitting multiple sclerosis (MS-RR) and secondary progressive multiple sclerosis (MS-SP) patients without consideration of gender;

FIG. 8 is a box plot which shows the separation of male, female and MS subtypes based on their normalized suppression of COUPTF-II; and

FIG. 9 is a box plot which shows that there is no inherent gender-based difference in COUPTF-II independent of disease activity.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

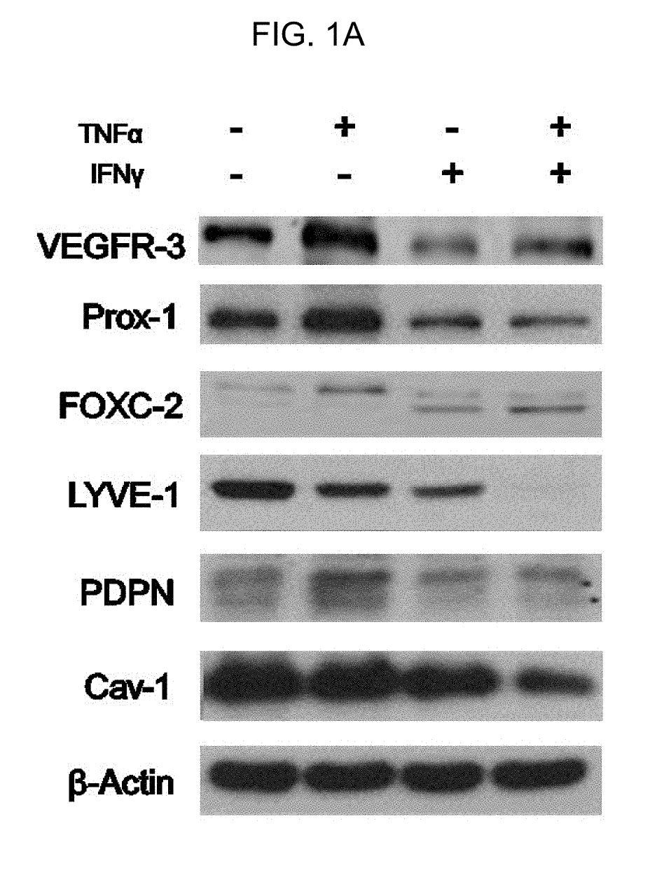

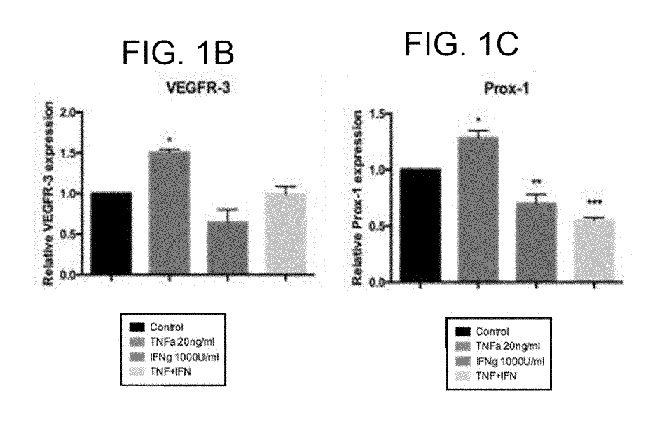

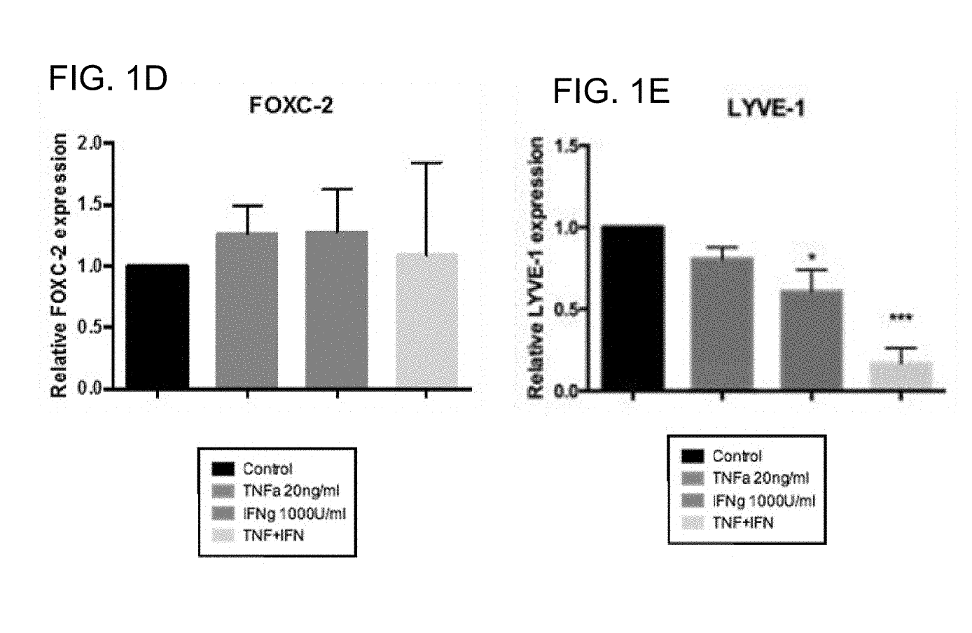

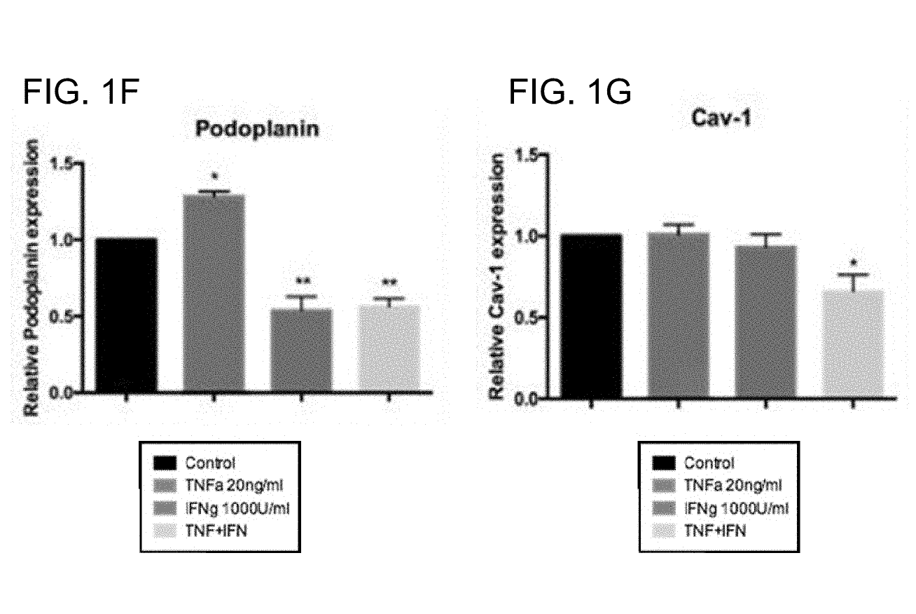

Turning now to FIGS. 1A-9, a brief description concerning the various components of the present invention will now be briefly discussed.

The presently claimed invention involves the use of western blotting (WB), fluorescence-activated cell sorting (FACS) or enzyme-linked immunosorbent analysis (ELISA), for example, to analyze, diagnose, stage neurodegenerative and dementia diseases and to gauge the therapeutic efficacy of treatments for these diseases using a novel panel of circulating soluble proteins released by brain endothelial cells in ‘microparticles’ which are secreted ‘caveolar’ membrane-microdomains. The inventors have experimentally confirmed that the circulating depot of these biomarkers are initially liberated in small (<1 μm) microparticles which in this setting are brain endothelial membrane-derived shed caveolae (cholesterol-enriched membrane ‘blebs’. Microparticles are continuously released by endothelial cells and the rate of microparticle release is increased by exposure to conditions which are found in chronic inflammatory states including multiple sclerosis (MS) and Alzheimer's disease (AD). The inflammatory milieu in these conditions is characterized by an increased abundance of inflammatory ‘cytokines’, inflammatory activator proteins which modulate a wide variety of responses in neurodegenerative disease, but also in many other chronic inflammatory states e.g. osteo- and rheumatoid arthritis, lupus, Crohn's disease, ulcerative colitis, graft vs. host disease, transplant injury and asthma. Neurovascular stress (like that which drives microparticle release) is now recognized as a significant and important contributor to the activity of neurodegenerative diseases. The complement of proteins in this diagnostic panel which until recently, have been mainly described as being expressed on lymphatic endothelial cells and lymphatic vessels, which are considered to be ‘outside’ of the central nervous system (CNS). Because lymphatics and neurolymphatics function to clear disease modifying proteins and mediators like cytokines and beta-amyloid, the suppression of lymphatics, measured as a diminution of lymphatic structural and lineage-determining transcription factors would contribute to lymphostasis/neurolymph-stasis which fails to clear these injurious substances from the CNS and intensify disease activity. Such markers may also reveal changes in these proteins during chronic inflammatory states outside of the CNS as well. The inventors also specifically anticipate that the neurolymphatic disturbances seen in MS would also be observed in Alzheimer's disease (AD) since amyloid-beta, one of the key biomarkers and causes of this condition appears to be drained at least in part through neurolymphatic pathways, therefore reductions in these markers would be anticipated to be depressed in this condition as well.

The inventors disclose proteins within this panel both as a non-centrifugable freely-circulating pool and also a pool which is associated with microparticles which can be harvested by 10,000 g centrifugation. Both pools are found in and seen to be modulated by cytokine-stimulation in culture supernatants from human and mouse brain endothelial cells as well as in both control and multiple sclerosis serum samples which also show differences in these markers. Because microparticles are cleared from the vascular space by the reticuloendothelial system within hours of the time they are formed, they appear to represent a circulating recent ‘snapshot’ of the surface of the vasculature which describes the inflammatory complexion of the vasculature. In this manner, sampling of this pool is very simple and relatively non-invasively accomplished, involving as little as a <50 μl blood sample. Recent testing indicates that an effective discriminative analysis of the relative serum abundance of the proteins in this panel can be accomplished with as little as 0.25 μl of serum, indicating that a fingertip draw could provide sufficient sample for analysis. In this manner, it would be possible to repeatedly measure these marker proteins in patients, potentially adopting scale technology currently used in diabetes testing approaches. This would allow simple, safe and repeated testing and tracking of a biomarker panel which may establish individual baseline profiles, measure responses to therapy and potentially anticipate disease intensifications. Additionally, these lymphatic biomarkers may also be tested using immunoanalysis (as described below) in plasma, cerebrospinal fluid, CSF, saliva, urine and tears and other biological fluids. In the event that patients show a reduced COUPTF-II level in addition to a suspected diagnosis of multiple sclerosis, they might be started earlier on biologic and immunomodulatory therapies e.g. natalizumab, vedolizumab and steroids. This is a critical advantage since it is widely known that earlier treatment to arrest disease activity can potently suppress the aggressive progression of the disease.

The release of microparticles is increased in response to inflammatory cytokine mediators, transferring these proteins into a circulating pool, which is analyzed as a ‘liquid biopsy’ of the vascular endothelial surface to provide important information on the state of activation and inflammation within the brain. The inventors have studied these markers and found that over storage, markers which are originally restricted to microparticles are liberated into the soluble, non-centrifugable fraction which simplifies their analysis.

Current tests for neurodegenerative disease, and for a great many other diseases, often rely on the robustness of a single biomarkers specificity and sensitivity. This means that any biomarker preferably varies closely with disease activity and preferably does not fluctuate independent of disease status, which could potentially invalidate the utility of the test. The use of multiple transcription factors, proteins which govern the expression of structural and signaling biomarkers provide information regarding how close to a particular cell lineage (or state of differentiation) a tissue may be under a set of conditions. In this approach, the COUP-TFII, a lymphatic endothelial fate-determining transcription factor, in conjunction with other lymphatic transcription factors may predict the ability of neurolymphatic networks to clear inflammatory stimuli. If lymphatic networks lose their appropriate differentiated structure/function changes in neurolymphatics could potentially diagnose different neurodegenerative diseases, predict ‘flares’ and gauge the efficacy of treatments to treat such diseases.

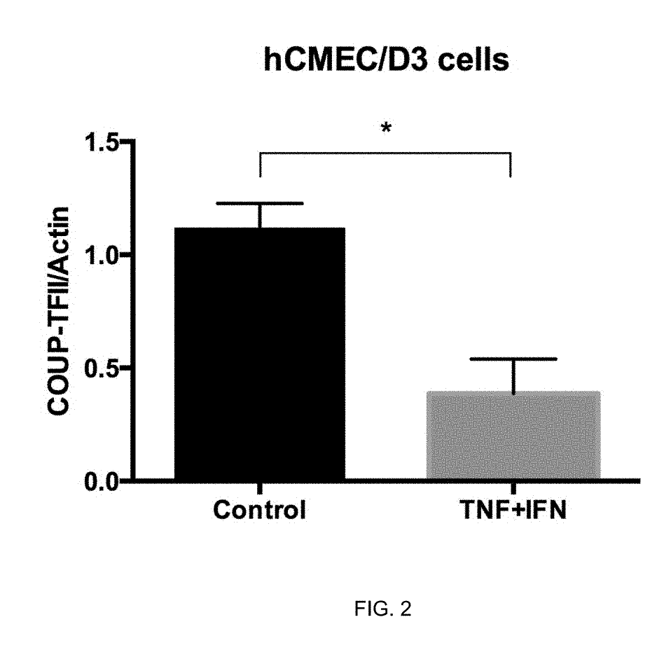

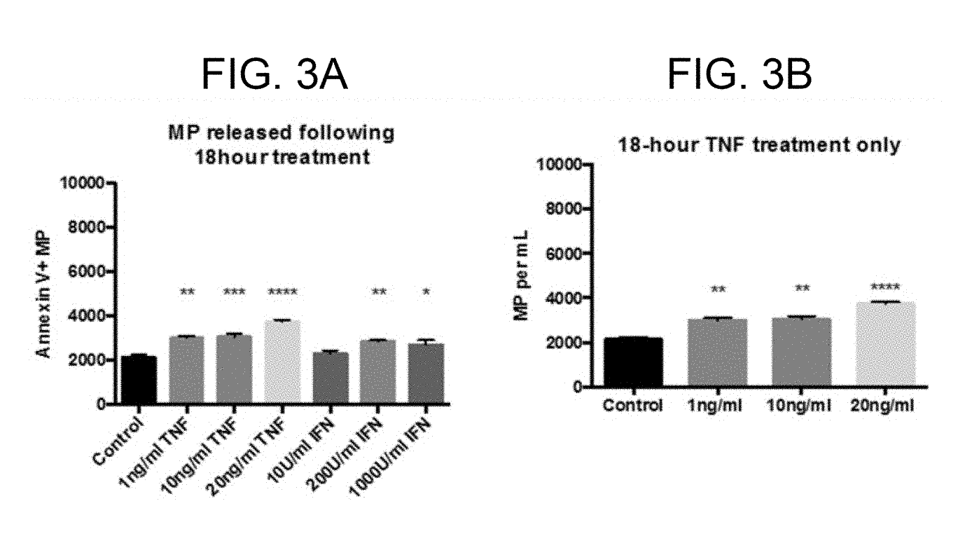

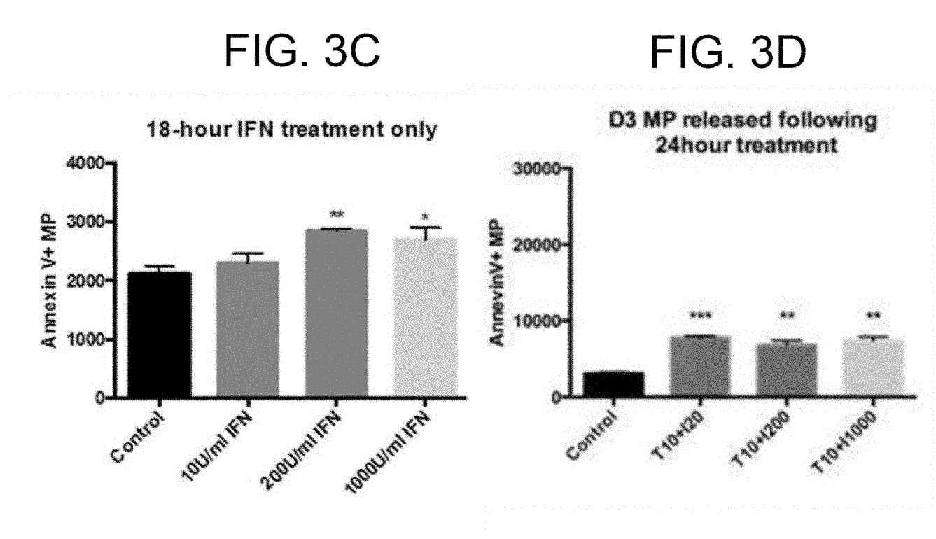

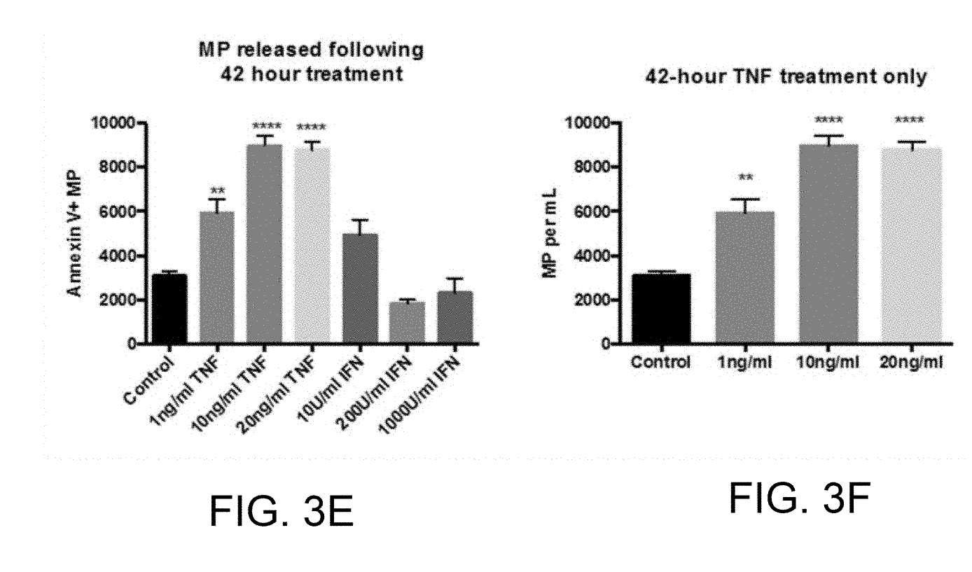

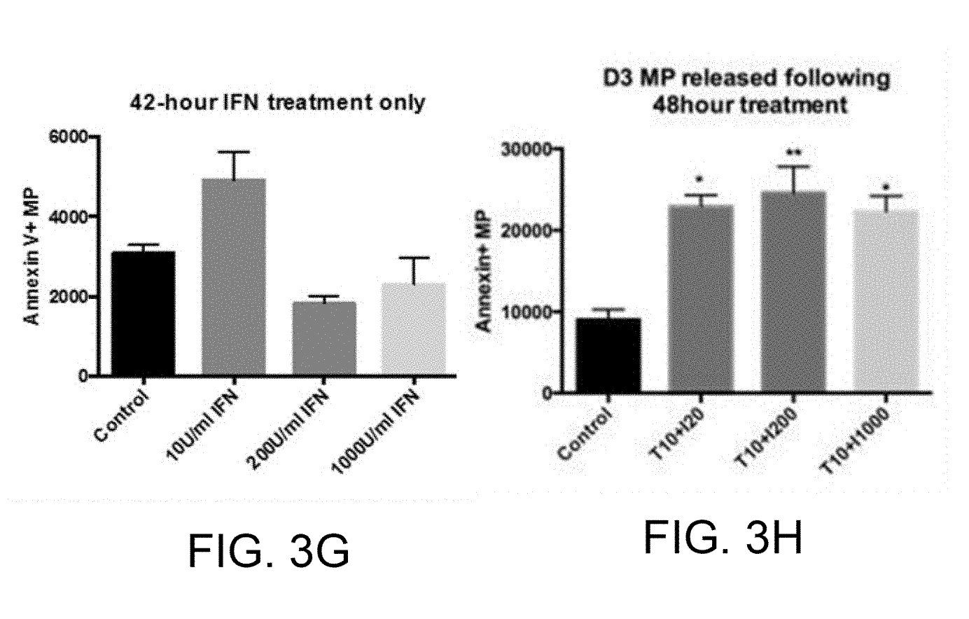

Current versions of this approach anticipate that suppression of transcription factors and their growth factor and structural protein targets would be useful analytes revealing evidence of brain endothelial stress induced by inflammatory cytokines (FIGS. 1A-2). The Inventors have evaluated the expression of these proteins in endothelium in response to inflammatory mediators and found that combined cytokine stresses (tumor necrosis factor-alpha/interferon-gamma) suppressed Prox-1, LYVE-1, podoplanin and caveolin-1 (FIG. 1A-1G). The same is true for the lymphatic fate determining transcription factor COUPTF-II (FIG. 2). Therefore, these studies suggest a compromised neurolymphatic capacity under strong cytokines challenges. Under these conditions, the inventors found that apical microparticles (AMP) are increasingly shed from the brain vascular endothelial surface (FIG. 3). These particles carry many of these factors with them which can provide information on the state of the vasculature within the brain.

As can be seen in this use of the disclosed invention, the expression of lymphatic proteins is detected in brain endothelial cells and can be increased by modest inflammation (TNF-a alone) but is apparently decreased by more powerful inflammation challenges (IFN-g or combined TNF-a/IFN-g treatments).

Turning to FIG. 2, another aspect of the invention is shown. This includes that COUP-TF-II is suppressed in response to inflammatory cytokine challenge Human brain endothelial cells express and downregulate COUPTF-II, a lymphatic fate determining transcription factor in response to cytokine (tumor necrosis factor-alpha+interferon-gamma) treatment after 24 h. This data demonstrates that the approach is informative in that these markers are transferred into the ‘mobile’ (liquid) phase and represents a pooled subset of the apical membrane. The analysis of these markers found in aqueous media permits evaluation of the originating source, e.g., the cytokine-inflamed endothelium.

Next, FIG. 3 shows release of apical microparticles (AMP) shedding by brain endothelial cells is time and cytokine concentration dependent. Endothelial cells continuously release MP and the rate of this release is increased by both TNF and IFN in a concentration dependent manner. This demonstrates that while NEBULA markers are associated with microparticles in plasma, samples of serum promote microparticle ‘fission’ confirmed by centrifugation recovery of caveolin-1 in plasma whereas this marker is also liberated in serum and is only found in the liquid phase. Similar recovery profiles are also seen for the lymphatic biomarkers LYVE-1, Prox-1 and VEGFR-3.

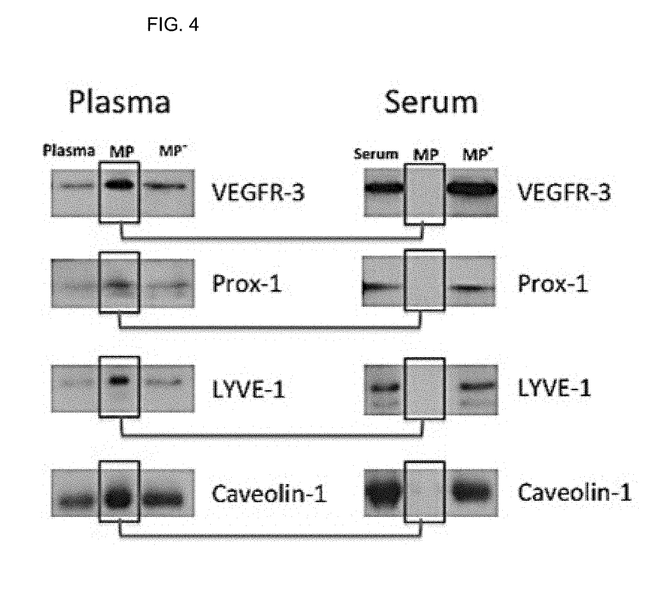

Shown in FIG. 4 is the release and progressive conversion of brain EMP associated lymphatic markers with MP fission. In the Plasma column on the left of the figure, the far left Plasma sub-heading also labeled “plasma” represents unfractionated plasma. The middle Plasma subheading, labeled “MP”, specifies an MP fraction isolated by 20,000×g centrifugation. The right Plasma subheading, labeled “MP”, denotes the fraction without microparticles. In the Serum column on the right of the figure, the far left Serum sub-heading also labeled “serum” represents unfractionated serum. The middle Serum subheading, labeled “MP”, specifies an MP fraction isolated by 20,000×g centrifugation. The right Serum subheading, labeled “ME”, denotes the fraction without microparticles. Boxes are drawn over each of the MP's for each protein tested in each of Plasma and Serum columns, to emphasis the differences in the two columns.

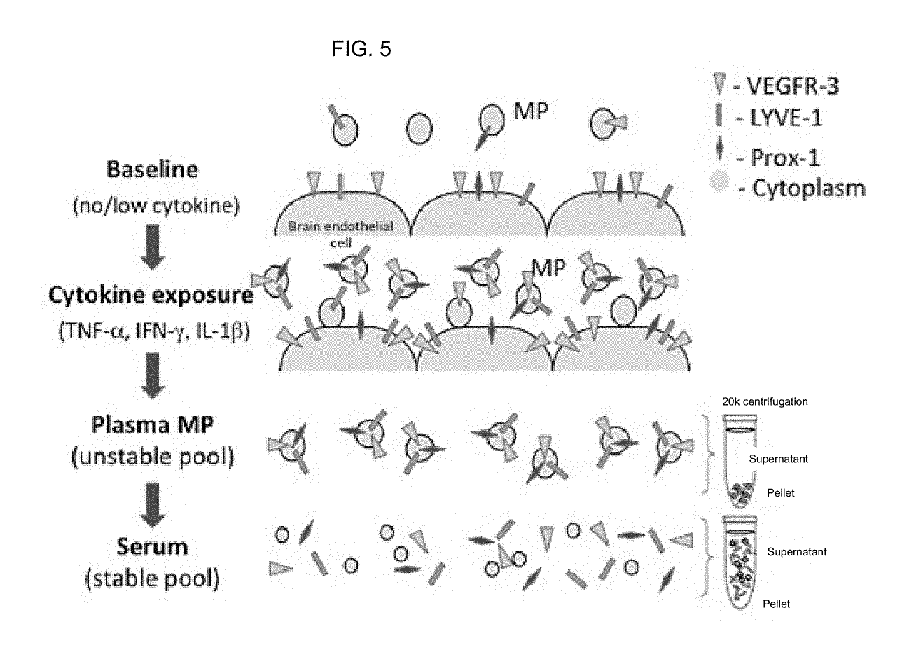

These data show that progressive microparticle fission quantitatively and substantially or effectively completely transfers these biomarkers into serum, (this fission is described schematically in FIG. 5). Therefore stored and frozen serum is a more superior, stable and representative analyte than plasma which should be employed in this analytical approach. Interestingly, as little as 0.25 μl of serum samples prepared in this manner provides sufficient material for biomarker testing, making this a highly robust approach.

FIG. 5 shows the progression from microparticle (MP) shedding by brain endothelial cells, where MPs containing neurolymphatic biomarkers are shed following exposure to cytokine. These MPs arrive immediately in the plasma pool which can be centrifuged; it is important to note that use of plasma microparticles could lead to varying sample recovery depending on the time of centrifugation after recovery. By comparison, serum represents a much more stable pool for analysis.

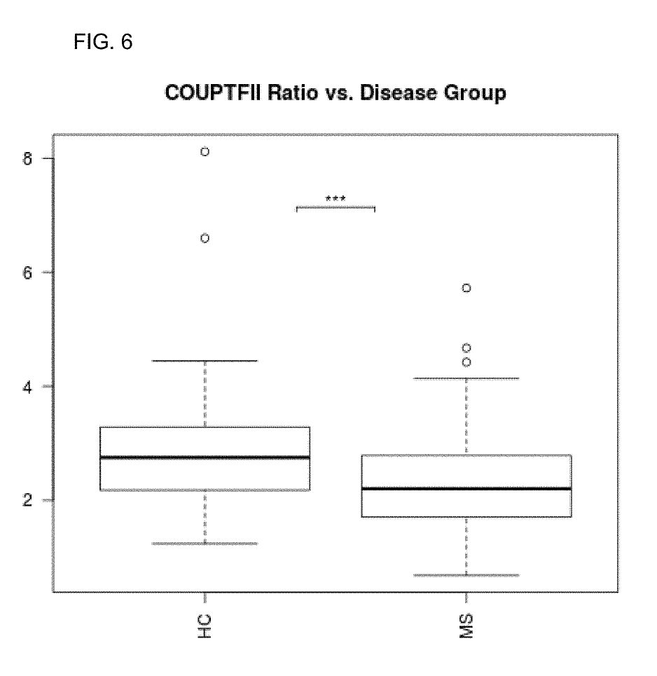

FIG. 6 shows the normalized ratio of the lymphatic fate-specifying transcription factor COUPTF-II (adjusted to total protein loading), which was found to be significantly different between healthy controls (HC) and MS patients (both RRMS (relapse remitting MS) and SPMS (secondary progressive MS)) considered as a single group) (***−p<0.001) when considered in aggregate, and separately for the RRMS (RR) and SPMS (SP) populations. These findings demonstrate that MS disease, irrespective of subgrouping, show a reduction in the expression of this lymphatic differentiation marker, consistent with a decrease in the normal level of lymphatic specification in MS which is also found in inflammatory cytokine stressed human brain endothelial cells. Significant reductions were similarly shown with at least FOXC2 and LYVE1 in both subgroups. (See Table 1). Additionally, PROX1 and Podoplanin showed significant reductions with MS and RR populations compared to the HC.

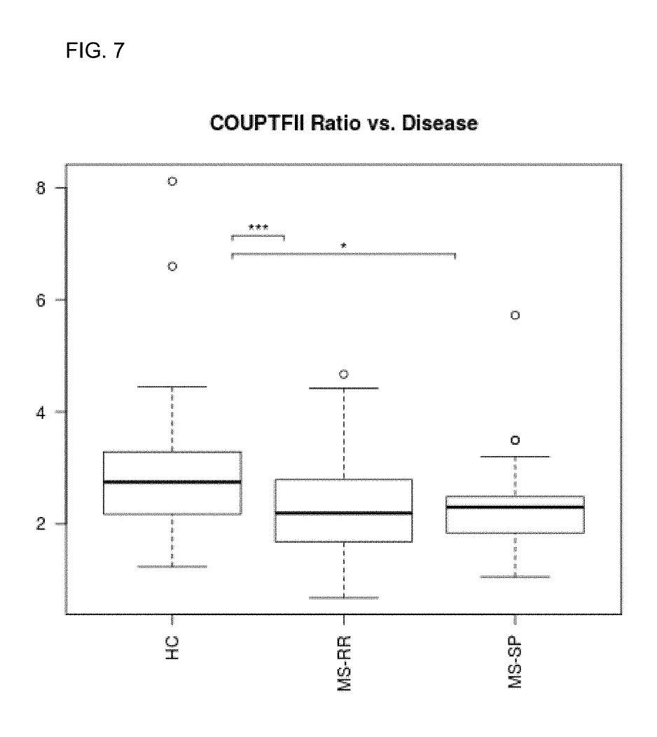

FIG. 7 shows various normalized serum dot blot results. The normalized ratio of the lymphatic fate-specifying transcription factor COUPTF-II (adjusted to total protein loading) was found to be significantly different between healthy controls (HC) and different groups of MS patients (***−p,0.001 for MS-RR and *−p<0.05 for MS-SP). These data do not factor in gender, only disease diagnosis. These findings show that in aggregate both disease forms show a reduction in the expression of this lymphatic differentiation marker consistent with a decrease in the normal level of lymphatic specification which is found in cytokine stressed brain endothelial cells.

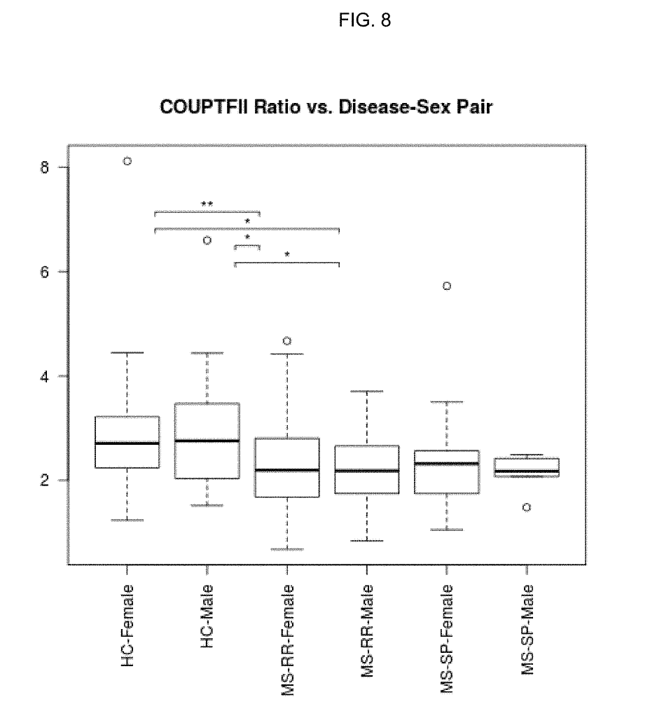

Turning to FIG. 8, the ratio of the lymphatic fate-specifying transcription factor COUPTF-II was found to be significantly different between healthy female controls (HC-female) and female RR MS patients (MS-RR female) (**−p<0.01) and male RR MS patients (MS-RR male). The ratio of the lymphatic fate-specifying transcription factor COUPTF-II was found to be significantly different between healthy male controls (HC-male) and male RR MS patients (MS-RR male) (**−p<0.01) and male RR MS patients (MS-RR male). These findings may show that each disease form shows a reduction in the expression of this lymphatic differentiation marker consistent with a decrease in the normal level of lymphatic specification which is found in cytokine stressed brain endothelial cells. Further refinement using other lymphatic markers, including for example, PROX1, FOXC2, LYVE1, and Podoplanin, improves the specificity and selectivity of this approach. That is, multiple markers, each with high levels of significance, dramatically increases the elucidation and discriminative capability of the diagnostic test. The method for analyzing (preferably soluble serum) samples from patients with potentially neurovascular or neurodegenerative disease may include obtaining a clinical or in vitro sample, performing enzyme-linked immunosorbent analysis, performing FACS or WB analysis of these samples using multiple analyte approach, and/or performing applying principal component analysis to evaluate sample based on treatment protocol or diagnostic impression. For each condition being evaluated (MS or Alzheimer's etc.), a qualitative and/or quantitative range of absolute or comparative values is preferably established for each of the analytes being tested. Preferably, two analytes are tested, more preferably three analytes are tested, even more preferably four or more analytes are tested to determine diagnosis or prognosis of condition.

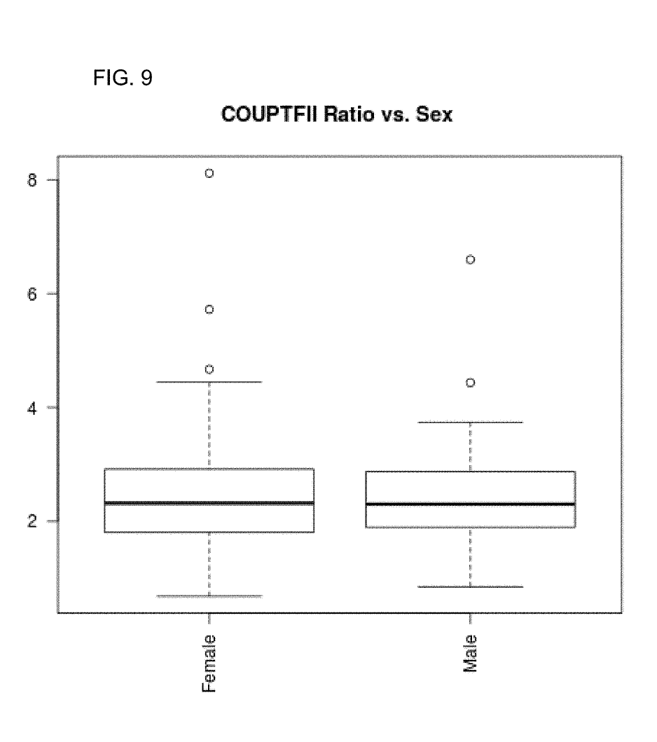

Shown in FIG. 9, the normalized intensity of the lymphatic fate-specifying transcription factor COUPTF-II was not significantly different between males and females.

The inventors have evaluated changes in ‘chicken ovalbumin upstream promoter transcription factor II’ (COUP-TF II), a novel lymphatic-selective brain-lymphatic (or ‘neurolymphatic’) transcription factor. The inventors' results suggest that this marker is discriminative for MS disease activity and type (FIGS. 6-9). Used in combination with other NEBULA biomarkers e.g. Prospero homeobox transcription factor-1 (Prox-1), Forked winghead transcription factor-2 (Fox2), as well as the lymphatic vascular endothelial hyaluronic acid receptor (LYVE-1), vascular endothelial growth factor receptor-3 (VEGFR-3), podoplanin.

The inventors disclose a single or multi marker develop diagnostic/prognostic test for MS. These approaches are also applicable to all other chronic inflammatory diseases which are characterized by inflammatory cytokine elevation (e.g., Crohn's disease, ulcerative colitis, arthritis, lupus, and graft vs. host disease and transplant rejection as a partial list).

Preliminary studies used the binding of Annexin V and expression of caveolin-1 to describe microparticles as the initial location of these markers; ultimately microparticle ‘fission’ liberates these proteins from centrifugable particles. Therefore, while the inventors have identified microparticles as a transitional intermediate location for neurolymphatic markers, the continuous fission of microparticles into microsomes transfers these markers into a pool which does not require centrifugation. Therefore it is ultimately more simple and more reliable to use either non-centrifuged or frozen-stored serum samples. It is theoretically possible that an immediate centrifugal isolation and concentration of microparticles from serum could yield an even more concentrated analyte, however so far the inventors' experimental results indicate this is not necessary. This test is a similar, but apparently more reliable and interpretable circulating biomarkers similar to other ‘soluble’ biomarkers e.g. soluble ICAM-1, VEGF, etc.

Evaluation of neurovascular biomarkers of lymphatic activation (NEBULA), provide important ‘information’ on the state of endothelial activation which so far has been validated with bona fide human endothelial cell models with inflammatory cytokines to yield an analytical testing method. The inventors have found that some protein NEBULA distinguish health from neurodegenerative disease, and have sub-grouped different forms of MS (RRMS vs. SPMS) using this approach.

Because several NEBULA markers can be simultaneously analyzed, each individual component of the multi-analyte panel contributes to the creation of an algorithm known as a ‘principal component analysis’ (PCA), which provides greater predictive power over an individual specie. This approach has been enabled by the inventors and is adaptable for automated scoring and interpretation of these test results. This method is immediately useful to interpret central nervous system inflammation, and may be applied to also evaluate other forms of systemic inflammation. Referring to Table 1, COUPTF-II, FOXC2, and LYVE1, for example, each appear to be individually predictive for MS disease and type. By evaluating for reduction in multiple or each of the three neurolymphatic biomarkers, for example, the testing algorithm becomes even more predictive. Further, referring to Table 1, the inventors found that the normalized levels of COUPTF-II, PROX1, FOXC2, LYVE1, and Podoplanin are each significantly lower in serum compared of MS individuals compared to healthy controls. Such a single lower serum level is indicative that that the patient has one of the forms of multiple sclerosis, either MS-RR or SP-MS. Multiple lower serum levels dramatically increases the reliability of the diagnosis.

The findings disclosed support and validate a concept that brain endothelial cells basally express apparently all known lymphatic endothelial cell biomarkers, many of which are recoverable and detectable within microparticles shed from inflammation-activated endothelium. This provides a liquid snapshot of the luminal surface of the brain vasculature during inflammatory phenomena and constitutes a novel means of evaluating ex vivo the level of inflammatory activation of the neurovasculature.

TABLE 1

| Mean | HC | MS | RR | SP |
|---|---|---|---|---|
| Intensity | (n = 60) | (n = 176) | (n = 150) | (n = 26) |
| PROX1 | | ↓, p = 0.0298 | ↓, p = 0.030 | ↓, p = 0.20 |
| | | vs. HC | vs. HC | (n.s. vs. HC) |
| COUP-TFII | | ↓, p = 0.00035 | ↓, p = 0.00055 | ↓, p = 0.00037 |
| | | vs. HC | vs. HC | vs. HC |
| FOXC2 | | ↓, p = 0.010 | ↓, p = 0.0427 | ↓, p = 0.00015 |
| | | vs. HC | vs. HC | vs. HC |
| | | | | ↓, p = 0.0042 |
| | | | | SP vs. RR |
| LYVE1 | | ↓, p = 0.0058 | ↓, p = 0.010 | ↓, p = 0.0094 |
| | | vs. HC | vs. HC | vs. HC |
| Podoplanin | | ↓, p = 0.02 | ↓, p = 0.0128 | p = 0.46 |
| | | vs. HC | vs. HC | (n.s. vs. HC) |

Table 1, above, shows which NEBULA markers are significantly reduced (↓) in serum, in which conditions (MS, RR, SP) the statistical significance (p=0.0 etc.) and the group to which the statistic is referring (vs. HC, etc). For each of PROX1, COUP-TFII, FOXC2, LYVE1, and Podoplanin, the scan density values for each of RR, SP, and MS (combined RR and SP) are compared to the scan density values for HC. The direction of change with respect to HC and the associated p values are shown for each of the conditions and targets—with a down arrow indicating a decrease. As shown, most markers are significant for the given condition compared to healthy controls. FOXC2 was able to discriminate RR vs. SP MS groups.

TABLE 2

| Mean | HC | RR | SP |
|---|---|---|---|
| Intensity | (n = 60) | (n = 150) | (n = 26) |
| PROX1 | 178481.4 | 175132 | 175153 |
| COUP-TFII | 215266 | 209868 | 208865 |

Table 2 shows the quantitative values for mean intensity of scan density for HC, RR, and SP for the PROX1 and COUP-TFII markers. Though the percent differences are small, the significance, as shown in Table 1, of MS, RR, and SP for COUP-TFII and MS and RR for PROX1 is very strong.

The analysis of the lymphatic endothelial (or neurolymphatic) fate-specifying transcription factor COUP-TFII (see FIGS. 6-9) in conjunction with analysis of other NEBULA markers, including, for example lymphatic endothelial growth factor (VEGFR-3) receptors, adhesive (Reelin, podoplanin, alpha9 integrin) and guidance/matrix proteins lymphatic vascular endothelial hyaluronic acid receptor represents a collective, multi-component biomarker which has enormous commercial value to companies developing tests for neurodegenerative diseases e.g. MS and AD which could provide a mechanism-based test revealing cerebrovascular stress. This approach might be a secondary test to confirm or validate magnetic resonance imaging (MRI) results, or a primary test for these conditions subsequent to appropriate testing. Drug companies testing the efficacy of biologics and immune modulators for human therapy would be interested in the availability of a simple test to explore how and which symptoms of disease track with which markers. The approach also uses an unbiased computer interpretation to create the PCA test and has higher through-put than film reading in some respects.

A testing kit based on this invention, would preferably include one or more of an immobiling phase e.g. a piece of nitrocellulose, to which a sample would be applied. This would be dried and blocked with 5% bovine serum albumin for 1 h and then incubated with an anti-NEBULA antibody, such as an anti-COUPTF-II antibody, at a dilution of, for example, 1:1000 in 0.1% bovine serum albumin in saline for 1 h, for example. This would preferably be washed 3 times in in 0.1% bovine serum albumin in saline and then incubated in a secondary antibody directed against the anti-NEBULA antibody conjugated to horseradish peroxidase, for example, for 30 minutes. After preferably washing 3× in 0.1% bovine serum albumin in saline this would be reacted with tetra-methyl benzidine/0.001% hydrogen peroxide for up to 10 mins or using enhanced chemiluminescence (ECL) with x-ray film. This would be compared to a group of controls or a pooled control human serum sample and the signal (absorbance/optical density) compared statistically. This is a very inexpensive approach which could be produced in several formats adaptable for clinical or at home use. Variations on the ingredients and steps of the testing kit above which would be obvious to those of ordinary skill in the art are also included in the presently claimed invention. Although a single NEBULA may be tested with each kit, preferably the kit will include appropriate supplies for testing multiple NEBULA.

The disclosed embodiments and variations of the present invention have many advantages. Just some of the advantages are described herein. Currently, MRI is the most accurate imaging test for the diagnosis and staging of CNS neurodegenerative diseases. Behavioral and clinical tests also use effective disability scoring (EDSS) which is highly subjective. Additional unbiased and mechanism based testing methods are still needed which can exploit sensitive markers. The use of a combined marker system is much more highly powered and has better statistical utility. The sample that would be analyzed in these tests is a small (<50 μl) blood/serum sample which could be frozen-stored, shipped to a test facility without requiring immediate access to an MRI unit. This approach would lower costs and greatly reduce needs for patient travel and allow for more frequent testing at lower costs. Such samples could be obtained on demand, analyzed using relatively inexpensive supplies and stored frozen until analyzed in clinical labs.

The inventors' disclosed diagnostic method is far less invasive, and requiring only 0.25 μl per protein dot blot, can be easily performed on the side of a routine blood tests, especially for populations who may be suspected to be at risk. The use of the disclosed invention can provide a framework to create an initial snapshot of an individuals' microparticle profile which, inter alia, provides opportunities to compare with later samples and determine how disease is progressing in patients, or to show improvement with therapy to provide an inexpensive and accessible ‘personalized’ medicine approach, or to compare with population averages to allow for early diagnosis or confirmation diagnosis.

Though various advantages and advantageous features have been described, the present invention does not require all of the advantageous features or all of the advantages to be included into every embodiment.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method for diagnosing a chronic inflammatory disease in a patient comprising:
testing a biological sample from the patient for a level of a first neurovascular biomarker of lymphatic activation; and
diagnosing the patient as having the chronic inflammatory disease if the tested level of the first neurovascular biomarker of lymphatic activation is less than a first normal level.

2. The method of claim 1, wherein the chronic inflammatory disease is one of multiple sclerosis, Alzheimer's disease, osteoarthritis, rheumatoid arthritis, lupus, Crohn's disease, ulcerative colitis, graft vs. host disease, transplant injury, and asthma.

3. The method of claim 1, wherein the chronic inflammatory disease is a brain endothelial related disease.

4. The method of claim 3, wherein the brain endothelial related disease is one of multiple sclerosis and Alzheimer's disease.

5. The method of claim 1 wherein the first neurovascular biomarker of lymphatic activation is one of chicken ovalbumin upstream promoter transcription factor II (COUPTF-II), Prospero homeobox transcription factor-1 (Prox-1), Forked winghead transcription factor-2 (Fox2), lymphatic vascular endothelial hyaluronic acid receptor (LYVE-1), vascular endothelial growth factor receptor-3 (VEGFR-3), and podoplanin.

6. The method of claim 1 further comprising testing the biological sample from the patient for a level of a second neurovascular biomarker of lymphatic activation, wherein the second neurovascular biomarker of lymphatic activation is different from the first neurovascular biomarker of lymphatic activation, and diagnosing the patient as having the chronic inflammatory disease if the tested level of both the first neurovascular biomarker of lymphatic activation is less than the first normal level and the second neurovascular biomarker of lymphatic activation is less than a second normal level.

7. The method of claim 6 wherein the second neurovascular biomarker of lymphatic activation is one of chicken ovalbumin upstream promoter transcription factor II (COUPTF-II), Prospero homeobox transcription factor-1 (Prox-1), Forked winghead transcription factor-2 (Fox2), lymphatic vascular endothelial hyaluronic acid receptor (LYVE-1), vascular endothelial growth factor receptor-3 (VEGFR-3), and podoplanin.

8. The method of claim 6 further comprising the step of diagnosing the patient as having the chronic inflammatory disease if the tested level of either the first neurovascular biomarker of lymphatic activation is less than the first normal level or the second neurovascular biomarker of lymphatic activation is less than the second normal level.

9. The method of claim 1 further comprising
testing the biological sample from the patient for a level of a second neurovascular biomarker of lymphatic activation, wherein the second neurovascular biomarker of lymphatic activation is different from the first neurovascular biomarker of lymphatic activation,
testing the biological sample from the patient for a level of a third neurovascular biomarker of lymphatic activation, wherein the third neurovascular biomarker of lymphatic activation is different from the first and the second neurovascular biomarker of lymphatic activation, and
diagnosing the patient as having the chronic inflammatory disease if the tested level of each of the first neurovascular biomarker of lymphatic activation is less than the first normal level, the second neurovascular biomarker of lymphatic activation is less than a second normal level, and the third neurovascular biomarker of lymphatic activation is less than a third normal level.

10. The method of claim 9 further comprising diagnosing the patient as having the chronic inflammatory disease if the tested level of at least two of the first neurovascular biomarker of lymphatic activation is less than the first normal level, the second neurovascular biomarker of lymphatic activation is less than the second normal level, and the third neurovascular biomarker of lymphatic activation is less than the third normal level.

11. The method of claim 1 further comprising
testing the biological sample from the patient for a level of a second neurovascular biomarker of lymphatic activation, wherein the second neurovascular biomarker of lymphatic activation is different from the first neurovascular biomarker of lymphatic activation,
testing the biological sample from the patient for a level of a third neurovascular biomarker of lymphatic activation, wherein the third neurovascular biomarker of lymphatic activation is different from the first and the second neurovascular biomarker of lymphatic activation,
testing the biological sample from the patient for a level of a fourth neurovascular biomarker of lymphatic activation, wherein the fourth neurovascular biomarker of lymphatic activation is different from the first, the second, and the third neurovascular biomarker of lymphatic activation, and
diagnosing the patient as having the chronic inflammatory disease if the tested level of each of the first neurovascular biomarker of lymphatic activation is less than the first normal level, the second neurovascular biomarker of lymphatic activation is less than a second normal level, the third neurovascular biomarker of lymphatic activation is less than a third normal level, and the fourth neurovascular biomarker of lymphatic activation is less than a fourth normal level.

12. The method of claim 11 further comprising diagnosing the patient as having the chronic inflammatory disease if the tested level of at least three of the first neurovascular biomarker of lymphatic activation is less than the first normal level, the second neurovascular biomarker of lymphatic activation is less than the second normal level, the third neurovascular biomarker of lymphatic activation is less than the third normal level, and the fourth neurovascular biomarker of lymphatic activation is less than the fourth normal level.

13. The method of claim 1 wherein the biological sample is one of blood, serum, plasma, cerebrospinal fluid, saliva, urine, tears.

14. The method of claim 1 wherein the biological sample is serum.

15. A method for diagnosing and treating one of a chronic inflammatory disease comprising
testing a biological sample from the patient for a level of a first neurovascular biomarker of lymphatic activation;
diagnosing the patient as having the chronic inflammatory disease if the tested level of the first neurovascular biomarker of lymphatic activation is less than a first normal level; and
administering a therapeutic to the patient for the chronic inflammatory disease.

16. The method of claim 15 wherein the chronic inflammatory disease is one of multiple sclerosis, Alzheimer's disease, osteoarthritis, rheumatoid arthritis, lupus, Crohn's disease, ulcerative colitis, graft vs. host disease, transplant injury, and asthma.

17. The method of claim 15 wherein the therapeutic is one of a biologic and a immunotherapy.

18. The method of claim 15 wherein the therapeutic is one of natalizumab, vedolizumab, and steroids

19. A kit for carrying out the testing of the biological sample in claim 1.

20. The kit of claim 19 further comprising an immobilizing phase and an anti-first neurovascular biomarker of lymphatic activation antibody.
